SEQUENCE LISTING (10 sequences)

SEQ ID NO: 1 - protein, 24 aa, Artificial Sequence
  Description of Artificial Sequencehuman relaxin-2, human gene-2 relaxin (rhRLX), A chain, positions 162-185
ZLYSALANKCCHVGCTKRSLARFC

SEQ ID NO: 2 - protein, 24 aa, Artificial Sequence
  Description of Artificial Sequencerat relaxin-1, A chain, positions 163-186
QSGALLSEQCCHIGCTRRSIAKLC

SEQ ID NO: 3 - protein, 24 aa, Artificial Sequence
  Description of Artificial Sequencerat relaxin-3, A chain, positions 117-140
DVLAGLSSSCCEWGCSKSQISSLC

SEQ ID NO: 4 - protein, 29 aa, Artificial Sequence
  Description of Artificial Sequencehuman relaxin-2, human gene-2 relaxin (rhRLX), B chain, positions 25-53
DSWMEEVIKLCGRELVRAQIAICGMSTWS

SEQ ID NO: 5 - protein, 35 aa, Artificial Sequence
  Description of Artificial Sequencerat relaxin-1, B chain, positions 23-57
RVSEEWMDQVIQVCGRGYARAWIEVCGASVGRLAL

SEQ ID NO: 6 - protein, 27 aa, Artificial Sequence
  Description of Artificial Sequencerat relaxin-3,B chain, positions 24-50
RPAPYGVKLCGREFIRAVIFTCGGSRW

SEQ ID NO: 7 - DNA, 21 bp, Artificial Sequence
  Description of Artificial Sequencerelaxin-3 specific forward primer
tgcggaggctcacgatggcgc

SEQ ID NO: 8 - DNA, 21 bp, Artificial Sequence
  Description of Artificial Sequencerelaxin-3 specific reverse primer
atagctgacagcaggttggac

SEQ ID NO: 9 - DNA, 24 bp, Artificial Sequence
  Description of Artificial SequenceLGR7 forward primer
gtgtatccttttcggtgtttaagg

SEQ ID NO: 10 - DNA, 24 bp, Artificial Sequence
  Description of Artificial SequenceLGR7 reverse primer
gaataagaattgagtctagatgac

(19) United States
(12) Patent Application Publication
(10) Pub. No.: US 2006/0264367 A1
(43) Pub. Date: Nov. 23, 2006

(54) Prevention of fibrosis following cardiac injury

(75) Inventors: Chrishan Surendran Samuel, Glen Waverley (AU); Ross Alexander David Bathgate, Brunswick West (AU); Geoffrey William Tregear, Hawthorn (AU); Xiao-Jun Du, Glen Huntly (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Howard Florey Institute, Parkville (AU); Baker Medical Research Institute, Melbourne (AU)

(21) Appl. No.: 11/133,763

(22) Filed: May 21, 2005

(51) Int. Cl. A61K 38/17

(52) U.S. Cl. 514/12

(57) ABSTRACT

A method for treating cardiac fibrosis resulting from injury of a mammalian heart is described comprising the step of contacting a therapeutically effective amount of relaxin and/or an LGR7 activating agent with cardiac cells following the injury in an amount sufficient to reduce the fibrosis. Also described are methods for protecting the heart following an ischemic event, inhibiting the proliferation of activated fibroblasts and antagonising collagen secretion or deposition in a mammalian heart.

### FIELD OF THE INVENTION

The present invention relates to a method of inhibiting, ameliorating or reversing cardiac fibrosis.

### BACKGROUND OF THE INVENTION

Cardiac fibrosis is a hallmark of heart disease and is the result of a variety of structural changes that occur after pathological stimuli to the cardiovascular system (Judgutt B I (2003) Curr Drug Targets Cardiovasc Haematol Disord 3:1-30). The fibrosis in heart disease is characterized by a disproportionate accumulation of fibrillar collagen that occurs after myocyte death, inflammation, hypertrophy, and stimulation by a number of hormones, cytokines, and growth factors (See e.g., Weber K T (1989) J Am Coll Cardiol 13:1637-1652); Bishop J E, Lindahl G (1999) Cardiovasc Res 42:27-44; Lijnen P J). The proximal effector cells in this process are fibroblasts, which when activated to become myofibroblasts produce an excessive amount of collagen in response to inflammatory mediators, such as TGF-13 (Petrov W V, Fagard R H (2000) Mol Genet Metab 71:418-435) and angiotensin II (Ang II) (Lijnen P J, Petrov V V, Fagard R H (2000) Methods Find Exp Clin Pharmacol 22:709-723). Cardiac fibroblasts are the predominant source of synthesis of interstitial proteins and other myocardial components which have been implicated in heart failure by their effects on diastolic function and, indirectly, by effects on cardiac myocytes to cause or potentiate systolic dysfunction (Hess et al, Circ., 63:360-371 (1981); Villari et al, Am J. Cardiol., 69:927-934 (1992); Villari et al, JACC, 22:1477-1484 (1993); Brilla et al, Circ. Res., 69:107-115 (1991); and Sabbah et al, Mol. & Cell Biochem., 147:29-34 (1995)).

Acute ischemic damage to the heart is associated with a progression to congestive heart failure. In addition to the original damage, the process of remodelling results in an even greater scarring response than necessary to mitigate the original damage, and this remodelling takes place over many months following an ischemic event. The additional scarring leads to a compensatory response in the remaining tissue and an eventual hypertrophy and chronic failure.

Chronic damage leading to cardiomyopathy is also associated with heart failure. Cardiomyopathy occurs when the heart becomes abnormally enlarged, thickened and/or stiffened due to fibrosis. This condition is generally a progressive one and may be caused by a wide range of conditions, including hypertension, chronic diseases, alcoholism, viral diseases, and others. An affected heart may grow larger either by dilatation, hypertrophy, or both. Additionally, the fibrosis associated with cardiomyopathies leads to a lessening in function of the tissue and decreased contractility

Hypertension affects 15-20% of the adult population leading to structural remodeling of the left ventricular (LV) myocardium and eventually heart failure. Several studies have focused on the contribution of the extracellular matrix to cardiac diastole and systole function. Tyagi S C et al. J Cell Physiol. (1996)167:137-147. It is now well established that the fibrillar collagen network in the ECM is integral in providing the structural support for cardiomyocytes and coronary vessels, as it imparts the myocardium with physical properties and influences ventricular diastolic and systolic function. A pathological stimulus to this network leads to the development of cardiac fibrosis, an integral characteristic of hypertensive heart disease.

Despite the pathophysiological significance of fibrosis in the heart, no effective treatment strategies exist for treatment or prevention of this response in acute or chronic heart injury. Thus, therapies inhibiting fibroblast metabolism and proliferation, deposition of collagen, and other aspects of the remodelling response are needed for conditions associated with cardiac fibrosis.

Mature human relaxin is a hormonal peptide of approximately 6000 daltons known to be responsible for remodelling the reproductive tract before parturition, thus facilitating the birth process. This protein appears to modulate the restructuring of connective tissues in target organs to obtain the required changes in organ structure during pregnancy and parturition. See, Hisaw, F. L., Proc. Soc. Exp. Biol. Med., 23: 661-663 (1926); Schwabe, C., et al., Biochem. Biophys. Res. Comm., 75: 503-570 (1977); James, R. et al., Nature, 267: 544-546 (1977). A concise review of relaxin was provided by Sherwood, D. in The Physiology of Reproduction, Chapter 16, “Relaxin”, Knobil, E. and Neill, J., et al. (eds.), (Raven Press Ltd., New York), pp. 585-673 (1988). Circulating levels of relaxin are elevated for the entire nine months of pregnancy and drop quickly following delivery.

While predominantly a hormone of pregnancy, relaxin has also been detected in the non-pregnant female as well as in the male. Bryant-Greenwood, G. D., Endocrine Reviews, 3: 62-90 (1982) and Weiss, G., Ann. Rev. Physiol., 46:43-52 (1984). Three human gene forms have been identified for relaxin (H1, H2 and H3). Hudson, P., et al., Nature, 301: 628-631 (1983); Hudson, P., et al., The EMBO Journal, 3: 2333-2339 (1984); U.S. Pat. Nos. 4,758,516 and 4,871,670. and U.S. Pat. App. No. 20050026822. Only one of the gene forms (H2) has been found to be transcribed in corpus luteum. Another form (H3) is found primarily in the central nervous system, and it remains unclear whether the (H1) form is expressed at another tissue site, or whether it represents a pseudo-gene. When synthetic human relaxin (H2) and certain human relaxin analogs were tested for biological activity, the tests revealed a relaxin core necessary for biological activity as well as certain amino acid substitutions for methionine that did not affect biological activity. Johnston, et al., in Peptides: Structure and Function, Proc. Ninth American Peptide Symposium, Deber, C. M., et al. (eds.) (Pierce Chem. Co. 1985).

Relaxin has been found to bind specifically to receptors in the cardiac atria of both male and female rats. Cardiac atria from rat hearts respond directly to relaxin, through an increase in the rate of contraction in the isolated and spontaneously beating right atrium and an increase in the force of contraction in isolated and electrically paced left atrium. See U.S. Pat No. 5,166,191. Although relaxin had been described for increasing the force or rate of atrial contraction, e.g., for the treatment of brachycardia as described in U.S. Pat. No. 5,478,807, it had not been shown that relaxin would be effective to treat cardiac fibrosis following acute or chronic injury. The present invention addresses the use of relaxin for treatment of cardiac fibrosis.

### SUMMARY OF THE INVENTION

The present invention provides methods for treating diseases related to cardiac fibrosis following either chronic or acute injury. The methods generally comprise administering to an individual in need thereof a pharmaceutical formulation comprising a therapeutically effective amount of relaxin and/or an LGR7 activating agent to treat the resulting cardiac fibrosis. Reduction in fibrosis can be mediated by a decrease in fibroblast activation, myofibroblast differentiation, collagen synthesis, collagen deposition or matrix metaloproteinase (MMP) expression. The effect of relaxin and/or an LGR7 activating agent on these processes may be direct or indirect. The present invention overcomes shortcomings of the prior art by providing improved methods for reducing fibrosis and related pathologies in the heart as disclosed herein.

The present invention provides methods of use of relaxin to modulate cardiac fibrosis in the following ways: 1) by inhibiting the activation of fibroblasts, as assessed by expression of α-SMA (a marker of myofibroblasts); 2) by inhibiting the proliferation of activated myofibroblasts; 3) by antagonizing collagen deposition by activated myofibroblasts; and 4) by increasing collagen degradation via activation of MMPs. It is of interest to note that relaxin did not influence cardiac collagen expression under basal conditions when applied over a short-term period in vitro. Thus, relaxin only reduced cardiac collagen synthesis and accumulation in cardiac cells (and more specifically myofibroblasts) when stimulated by a number of factors.

Modes of administration, amounts of relaxin and/or an LGR7 activating agent administered, and specific formulations for use in the methods of the present invention, are discussed below.

In one specific aspect of the invention, relaxin can be used to treat cardiac damage following acute injury. The method includes the induction of a cardioprotective effect on surrounding tissue and a decrease in the level of tissue remodelling following acute cardiac injury. Reduction in remodelling will lead to a measurably smaller area of damage following the acute injury as can be shown and measured three months from injury, six months from injury, and twelve months from injury. The patient will also display an increased ejection fraction compared to an untreated control patient.

In one specific aspect of the invention, relaxin and/or an LGR7 activating agent can be used to treat cardiac damage following acute injury. The method includes the induction of a cardioprotective effect on surrounding tissue and a decrease in the level of tissue remodelling following acute cardiac injury. Reduction in remodelling will lead to a measurably smaller area of damage following the acute injury as can be shown and measured three months from injury, six months from injury, and twelve months from injury. The patient will also display an increased ejection fraction compared to an untreated control patient.

In another specific aspect of the invention, relaxin and/or an LGR7 activating agent can be used to treat cardiac fibrosis arising from chronic cardiac disease, such as cardiomyopathy, chronic injury due to hypertension (e.g., primary pulmonary hypertension), or congestive heart failure. Forms of cardiomyopathy that are amenable to treatment with the present invention include ischemic cardiomyopathy, idiopathic cardiomyopathy, dilated, hypertrophic cardiomyopathy, alcoholic cardiomyopathy, peripartum cardiomyopathy, and restrictive cardiomyopathy.

In a preferred embodiment, a therapeutically effective amount of relaxin and/or an LGR7 activating agent is delivered to a patient via systemic delivery. Systemic administration has the advantages of permitting a less invasive or noninvasive means for treating a patient following injury. In addition, systemic administration permits a physician to have greater control over drug administration, including frequency and dosage, without concern as to whether, for example, a locally administered drug is effectively releasing active ingredient or whether contents of an injection remain at the desired site. Systemic delivery includes, but is not limited to, intravenous injection, subcutaneous injection, pulmonary delivery, delivery via an implanted osmotic pump, and transdermal/percutaneous administration. In another embodiment, a therapeutically effective amount of relaxin and/or an LGR7 activating agent is delivered to a patient via local delivery. Local delivery provides relaxin to the area of damage directly, and-so is a more targeted method for reduction of fibrosis in a specific area of injury. Examples of such delivery include, but is not limited to, administration via the use of a stenting device, administration via a catheter (optionally attached to an osmotic pump), direct injection into or near the cardiac tissue, injection into the pericardium, a depot injection, and the like.

In some embodiments, the invention provides methods of treating cardiac fibrosis, comprising administering to a patient in need thereof a pharmaceutical formulation comprising pharmaceutically active relaxin in an amount effective to inhibit the activation of fibroblasts to myofibroblasts. In a specific embodiment, the relaxin is administered in an amount that measurably decreases expression of α-SMA in myofibroblasts. In some embodiments, the invention provides methods of treating cardiac fibrosis, comprising administering to a patient in need thereof a pharmaceutical formulation comprising pharmaceutically active relaxin in an amount effective to inhibit the differentiation of activated fibroblasts. In a specific embodiment, the relaxin is administered in an amount that decreases production of collagen by myofibroblasts. In some embodiments, the invention provides methods of treating cardiac fibrosis, comprising administering to a patient in need thereof a pharmaceutical formulation comprising pharmaceutically active relaxin in an amount effective to inhibiting the proliferation of activated fibroblasts.

In some embodiments, the invention provides methods of treating cardiac fibrosis, comprising administering to a patient in need thereof a pharmaceutical formulation comprising pharmaceutically active relaxin in an amount effective to antagonize collagen deposition by activated fibroblasts.

In some embodiments, the invention provides methods of treating cardiac fibrosis, comprising administering to a patient in need thereof a pharmaceutical formulation comprising pharmaceutically active relaxin in an amount effective to increase collagen degradation via activation of MMPs.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIG. 1. is a comparison of the amino acid sequence of rat relaxin-1, rat relaxin-3, and recombinant human gene-2 relaxin (rhRLX). Homologous amino acids are shaded, and the conserved relaxin receptor binding residues are boxed and shaded.

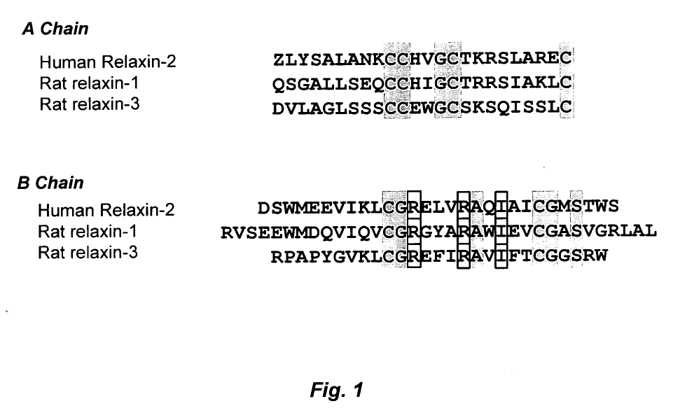

FIG. 2. is a bar graph showing MMP-2 expression for cells treated with either rhRLX (100 ng/ml) alone, TGF-β (1 ng/ml) alone, or TGF-β (1 ng/ml) and rhRLX (100 ng/ml) over 72 hours.

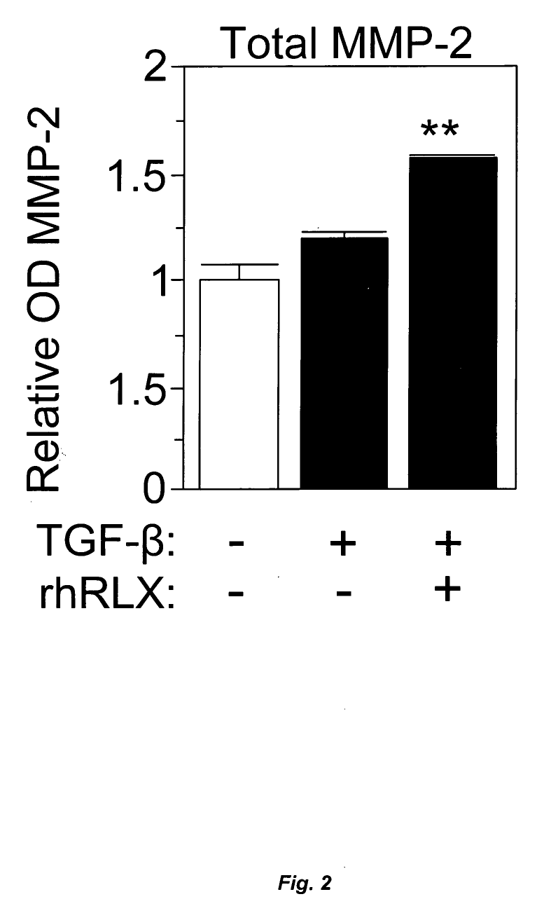

FIG. 3 is a series of bar graphs illustrating modulation of fibroblast proliferation by relaxin. FIG. 3A illustrates the effect of cardiac fibroblasts stimulated with Ang II (10⁻⁸ M and 10⁻⁷ M) in the absence of presence of rhRLX (100 ng/ml) over 72 hours and analyzed for cell proliferation by cell counts and [³H]-thymidine uptake. FIG. 3B illustrates the effects of IGF-I (0.1 μg/ml, 0.5 μg/ml, and 1.0 μg/ml) on cell proliferation in the absence or presence of rhRLX (100 ng/ml) over 72 hours.

FIG. 4 is a series of bar graphs illustrating the quantitation of the α-SMA products by densitometry presented as the mean±SE of relative OD α-SMA. *, P<0.05; and **, P<0.01 compared with values from untreated cells. †, P<0.05 compared with values from TGF-β-treated cells (4A) or Ang II-treated cells (4B). CTL, Control.

FIG. 5 illustrates the ability of rhRLX to decrease fibrosis in RLX(−/−) (FIG. 5A) and β₂-AR transgenic mice (FIG. 5B). P<0.05; and ***, P<0.001 compared with values from RLX+/+ and WT mice, respectively. †, P<0.05 compared with values from RLX−/− mice treated with vehicle alone. ††, P<0.01 compared with β₂-AR transgenic mice treated with vehicle alone. LV, Left ventricle.

FIG. 6 is a series of bar graphs illustrating collagen concentrations (μg/mg dry weight tissue) in the LV, RV, atria, and kidney of SHR-V, SHR-R and WKY rats (n=8-9 animals per group).

FIG. 7 is a series of bar graphs demonstrating densitometry scanning of the mean±SEM OD values of PCNA (FIG. 7A); α-SMA (FIG. 7B), and the latent forms of MMP-2 and -9 (FIG. 7C).

FIG. 8 is a series of bar graphs that illustrate mRNA levels of ANP (FIG. 8A) and βMHC (FIG. 8B), expressed as a ratio of gene to 18S values from the LV of 6-8 samples from WKY, SHR-V and SHR-R.

### DETAILED DESCRIPTION OF THE INVENTION

Before the present devices, cells and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a factor” refers to one or mixtures of factors, and reference to “the method of production” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention. Generally, conventional methods of cell culture, stem cell biology, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies : A Laboratory Manual: Portable Protocol NO. I, Cold Spring Harbor Laboratory (1998); and Harlow and Lane, Antibodies: A Laboratory Manual, Cold Spring Harbor Laboratory; (1988).

Although the present invention is described primarily with reference to cardiac fibrosis, it is also envisioned relaxin may play a significant role in resolving the fibrotic response following injury of other organ systems (e.g., renal). The present invention is intended to cover these uses of relaxin as well as the cardiovascular uses emphasized herein.

Definitions

As used in the present specification, the following words and phrases are generally intended to have the meanings as set forth below, except to the extent that the context in which they are used indicates otherwise.

The term “antibody” stands for an immunoglobulin protein which is capable of binding an antigen. Antibody as used herein is meant to include the entire antibody as well as any antibody fragments (e.g. F(ab′, Fab, Fv) capable of binding the epitope, antigen or antigenic fragment of interest. Preferred antibodies for use in the invention are immunoreactive or immunospecific for and therefore preferentially bind to LGR7.

Antibodies for LGR7 are preferably immunospecific—e.g., not substantially cross-reactive with related materials. The term “antibody” encompasses all types of antibodies, e.g. polyclonal, monoclonal, and those produced by the phage display methodology. Particularly preferred antibodies of the invention are monoclonal antibodies which have a relatively high degree of affinity for the target antigen.

The term “treatment” or “treating” means any therapeutic intervention in a mammal, including:
(i) prevention, that is, causing the clinical symptoms not to develop;
(ii) inhibition, that is, arresting the development of clinical symptoms; and/or
(iii) relief, that is, causing the regression of clinical symptoms.

The term “therapeutically effective amount” means a dosage sufficient to provide treatment for the disease state being treated. This will vary depending on the patient, the disease, the method of delivery, and the desired clinical outcome.

The term “relaxin” means human relaxin, including intact full length relaxin or a portion of the relaxin molecule that retains biological activity [as described in U.S. Pat. No. 5,023,321, preferably recombinant human relaxin (H2)] and other active agents with relaxin-like activity, such as Relaxin Like Factor (as described in U.S. Pat. No. 5,911,997 at SEQ ID NOS: 3 and 4, and column 5, line 27-column 6, line 4), relaxin and portions that retain biological activity analogs and portions that retain biological activity (as described in U.S. Pat. No. 5,811,395 at SEQ ID NOS: 1 and 2, and column 3, lines 16-40), and agents that competitively displace bound relaxin from a receptor. Relaxin can be made by any method known to those skilled in the art, preferably as described in U.S. Pat. No. 4,835,251 and in U.S. Pat. Nos 5,464,756.

The terms “LGR7” as used herein refers to a G protein-coupled receptor activated by relaxin H2, as described in Richard Ivell (2002) Science 295: 637-638.

The term “LGR7 activating agent” as used herein includes any molecules with the ability to activate LGR7 in the same manner as relaxin, i.e., activation that provides an antifibrotic response similar to relaxin upon administration as described in the methods herein. An LGR7 activating agent includes, but is not limited to, a small molecule, a peptidomimetic, a pharmacophore, or an activating anti-LGR7 antibody.

The term “pharmacophore” is used herein in an unconventional manner. Although the term conventional means a geometric and/or chemical description of a class or collection of compounds or groups, as used here the term means a compound that has a specific biochemical activity which activity is obtained by the 3-dimensional physical shape of the compound and the electrochemical properties of the atoms making up the compound. Thus, as used here the term “pharmacophore” is a compound and not a description of a collection of compounds which have defined characteristics. Specifically, a “pharmacophore” is a compound with those characteristics. More specifically, pharmacophores of the invention may, for example, mimic relaxin activity by interaction with an epitope of LGR7 to which relaxin binds. Thus, a pharmacophore of the invention has a shape (i.e., the geometric specifications) and electrochemical characteristics substantially as defined by the relaxin:LGR7 complex. The term pharmacophore covers peptides, peptide analogs and small molecules.

The Invention in General

Without being bound to a specific theory, the invention is based the novel finding that relaxin, through its G protein-coupled receptor (LGR7) (described in Hsu S Y et al. (2002) Science 295:671-674), mediates an antifibrotic effect and promotes the regenerative response following injury in cardiac tissue.

The antifibrotic properties of relaxin were investigated both ex vivo, in fibroblast cultures from neonatal rat cells, and in vivo using the relaxin-deficient (relaxin knockout) mouse, which develops an age-related progression of cardiac fibrosis (Du X J et al. (2003) Cardiovasc Res 57:395404), and the β₂-adrenergic receptor (β₂-AR) transgenic mouse, a more severe model of fibrotic cardiomyopathy (Gao X M, et al. (2003) Endocrinology 144:40974105). Specifically, the inventors have shown that relaxin H2 can prevent cardiac fibroblast proliferation, differentiation and activation in mice with existing cardiac fibrosis. Relaxin also inhibited the synthesis and deposition of collagen stimulated by profibrotic factors, which are known to be altered in human diseased states. This is the first finding that relaxin can treat cardiac pathologies with existing fibrosis and prevent further fibrotic damage in animals with existing cardiac damage.

Administration of a therapeutically effective amount of pharmaceutically active relaxin results in a decrease in remodeling in response to acute ischemic damage. Following administration of relaxin, the remodelling of the affected tissue is decreased by at least about 10%, at least about 20%, at least about 30%, at least about 50%, at least about 75%, or at least about 100%, or more when compared to a suitable control. Remodelling following an ischemic event can be measured by any method known in the art, including, but not limited to, magnetic resonance imaging (MRI), echocardiography (ECHO), electrocardiography (ECG), and magnetocardiography (MCG). Molecular markers or chemical markers such as MMPs and fragments of matrix proteins can also be used as indicators of a decrease in remodeling levels.

In some embodiments, methods are provided for promoting or enhancing cardiac tissue healing. Administration of an effective amount of a pharmaceutically active relaxin to an individual in need thereof promotes cardiac tissue healing by at least about 10%, at least about 20%, at least about 30%, at least about 50%, at least about 75%, or at least about 100%, or more when compared to a suitable control, e.g., the amount of necrotic tissue in the wound is decreased by at least about 10%, at least about 20%, at least about 30%, at least about 50%, at least about 75%, or at least about 100%, or more when compared to a suitable control. The efficacy of relaxin to promote cardiac tissue healing can be determined using any method known in the art. For example, the amount of necrotic tissue can be measured; and/or histochemical evaluation of a tissue biopsy can be conducted to determine the presence of and/or to measure the amount of tissue necrosis.

In some embodiments, methods are provided for reducing angiotensin II (AngII)-mediated mediated collagen secretion and/or deposition. Administration of an effective amount of a pharmaceutically active relaxin to an individual in need thereof reduces AngII-mediated collagen secretion and/or deposition by at least about 10%, at least about 20%, at least about 30%, at least about 50%, at least about 75%, or at least about 100%, or more when compared to a suitable control. Whether administration of relaxin reduces AngII-mediated collagen secretion and/or deposition can be determined using any method known in the art for measuring these parameters, including those described in Example 2.

In some embodiments, methods are provided for reducing Transforming Growth Factor-β (TGF-β) mediated collagen secretion and/or deposition. Administration of an effective amount of a pharmaceutically active relaxin to an individual in need thereof reduces TGF-β mediated collagen secretion and/or deposition by at least about 10%, at least about 20%, at least about 30%, at least about 50%, at least about 75%, or at least about 100%, or more when compared to a suitable control. Whether administration of relaxin reduces TGF-β mediated collagen secretion and/or deposition can be determined using any method known in the art for measuring these parameters, including those described in Example 2.

Cardiac Fibrosis

The methods of the present invention are suitable for treating an individual who has been diagnosed with a disease related to progressive cardiac fibrosis, who is suspected of having a disease related to progressive cardiac fibrosis, who is known to be susceptible and who is considered likely to develop a disease related to progressive cardiac fibrosis, or who is considered likely to develop a recurrence of a previously treated disease relating to progressive cardiac fibrosis.

Existing evidence demonstrates the association of fibrosis with the heart failure process in a variety of heart diseases, including those associated with both volume and pressure overload (Maron et al, Am. J. Cardiol., 35:725-739 (1975); Schwarz et al, Am. J. Cardiol., 42:661-669 (1978); Fuster et al, Circ., 55:504-508 (1976); Bartosova et al, J. Physiol., 200:285-295 (1969); Weber et al, Circ., 83:1849-1865 (1991); Schaper et al, Basic Res. Cardiol., 87:S1303-S1309 (1992); Boluyt et al, Circ. Res., 75:23-32 (1994); and Bishop et al, J. Mol. Cell Cardiol., 22:1157-1165 (1990)). In the setting of heart failure, fibrosis involves an increase in both fibroblast number and matrix deposition (Morkin et al, Am. J. Physiol., 215:1409-1413 (1968); Skosey et al, Circ. Res., 31:145-157 (1972); and Booz et al, Cardiovasc. Res., 30:537-543 (1995)), suggesting the importance of the fibroblast in the development of this condition. Cardiac fibroblasts are also the predominant source of synthesis of interstitial proteins and other myocardial components which have been implicated in heart failure by their effects on diastolic function and, indirectly, by effects on cardiac myocytes to cause or potentiate systolic dysfunction (Hess et al, Circ., 63:360-371 (1981); Villari et al, Am J. Cardiol., 69:927-934 (1992); Villari et al, JACC, 22:1477-1484 (1993); Brilla et al, Circ. Res., 69:107-115 (1991); and Sabbah et al, Mol. & Cell Biochem., 147:29-34 (1995)).

The treatment of the fibrotic cardiac disease state can be determined by measuring one or more diagnostic parameters indicative of the course of the disease, compared to a suitable control. For comparison with animal models, a “suitable control” is an animal not treated with relaxin, or treated with the pharmaceutical formulation without relaxin. In the case of a human subject, a “suitable control” may be the individual before treatment, or may be a human (e.g., an age-matched or similar control) treated with a placebo.

Cardiac fibrosis to be treated by the methods of the present invention may be due to a variety of diseases associated with cardiac fibroblast proliferation or the activation of extracellular matrix protein synthesis by cardiac fibroblasts. These diseases may be effectively treated in the present invention. Such diseases include aortic and mitral valvular regurgitation. In addition, cardiac hypertrophy, which is associated with many cardiac diseases, and often involves myocyte and fibroblast components, may be effectively treated in the present invention.

Heart failure is defined as the inability of the cardiac pump to move blood as needed to provide for the metabolic needs of body tissue. Decreases in pumping ability arise most often from loss or damage of myocardial tissue. As a result, ventricular emptying is suppressed which leads to an increase in ventricular filling pressure and ventricular wall stress, and to a decrease in cardiac output. As a physiological response to the decrease in cardiac output, numerous neuroendocrine reflexes are activated which cause systemic vasoconstriction, sympathetic stimulation of the heart and fluid retention. Although these reflex responses tend to enhance cardiac output initially, they are detrimental in the long term. The resulting increases in peripheral resistance increase the afterload on the heart and the increases in blood volume further increase ventricular filling pressure. These changes, together with the increased sympathetic stimulation of the heart, lead to further and often decompensating demands on the remaining functional myocardium.

Congestive heart failure, which is a common end point for many cardiovascular disorders, results when the heart is unable to adequately perfuse the peripheral tissues. According to recent estimates, there are about 4 million people in the United States diagnosed with this disease, and more than 50% of these cases are fatal within 5 years of diagnosis [Taylor, M. D. et al., Annual Reports in Med. Chem. 22, 85-94 (1987)].

Relaxin Formulations

Relaxin formulations suitable for use in the methods of the invention are pharmaceutical formulations comprising a therapeutically effective amount of pharmaceutically active relaxin, and a pharmaceutically acceptable excipient. The formulation is preferably injectable and most preferably designed for subcutaneous or intravenous injection.

Any known relaxin formulation can be used in the methods of the present invention, provided that the relaxin is pharmaceutically active. “Pharmaceutically active” relaxin is form of relaxin which results in decreased fibroblast activation, collagen secretion and/or collagen deposition when administered to an individual.

Relaxin may be administered as a polypeptide, or as a polynucleotide comprising a sequence which encodes relaxin. Relaxin suitable for use in the methods of the present invention can be isolated from natural sources, may be chemically or enzymatically synthesized, or produced using standard recombinant techniques known in the art. Examples of methods of making recombinant relaxin are found in various publications, including, e.g., U.S. Pat. Nos. 4,835,251; 5,326,694; 5,320,953; 5,464,756; and 5,759,807.

Relaxin suitable for use includes, but is not limited to, human relaxin, recombinant human relaxin, relaxin derived from non-human mammals, such as porcine relaxin, and any of a variety of variants of relaxin known in the art. Relaxin, pharmaceutically active relaxin variants, and pharmaceutical formulations comprising relaxin are well known in the art. See, e.g., U.S. Pat. Nos. 5,451,572; 5,811,395; 5,945,402; 6,780,836, 6,723,702, 5,166,191; and 5,759,807, the contents of which are incorporated by reference in their entirety for their teachings relating to relaxin formulations, and for teachings relating to production of relaxin. In general, recombinant human relaxin (rhRLX) is identical in amino acid sequence to the naturally occurring product of the human H2 gene, consisting of an A chain of 24 amino acids and a B chain of 29 amino acids.

Relaxin can be administered to an individual in the form of a polynucleotide comprising a nucleotide sequence which encodes relaxin. Relaxin-encoding nucleotide sequences are known in the art, any of which can be used in the methods described herein. See, e.g., GenBank Accession Nos. AF135824; AF076971; NM.sub.—006911; and NM.sub.—005059. The relaxin polynucleotides and polypeptides of the present invention can be introduced into a cell by a gene delivery vehicle. The gene delivery vehicle may be of viral or non-viral origin (see generally, Jolly, Cancer Gene Therapy (1994) 1:51-64; Kimura (1994) Human Gene Therapy 5:845-852; Connelly (1995) Human Gene Therapy 1:185-193; and Kaplitt (1994) Nature Genetics 6:148-153). Gene therapy vehicles for delivery of constructs including a coding sequence of a polynucleotide of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 415 731; WO 90/07936; WO 94/03622; WO 93/25698; WO 93/25234; U.S. Pat. No. 5, 219,740; WO 93/11230; WO 93/10218; Vile and Hart (1993) Cancer Res. 53:3860-3864; Vile and Hart (1993) Cancer Res. 53:962-967; Ram et al. (1993) Cancer Res. 53:83-88; Takamiya et al. (1992) J. Neurosci. Res. 33:493-503; Baba et al. (1993) J. Neurosurg. 79:729-735; U.S. Pat. No. 4,777,127; and EP 345,242.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

Gene delivery vehicles of the present invention can also employ parvovirus such as adeno-associated virus (AAV) vectors. Representative examples include the AAV vectors disclosed by Srivastava in WO 93/09239, Samulski et al. (1989) J. Vir. 63:3822-3828; Mendelson et al. (1988) Virol. 166:154-165; and Flotte et al. (1993) Proc. Natl. Acad. Sci. USA 90:10613-10617.

Also of interest are adenoviral vectors, e.g., those described by Berkner, Biotechniques (1988) 6:616-627; Rosenfeld et al.(1991) Science 252:431-434; WO 93/19191; Kolls et al. (1994) Proc. Natl. Acad. Sci. USA 91:215-219; Kass-Eisler et al. (1993) Proc. Natl. Acad. Sci. USA 90:11498-11502; WO 94/12649, WO 93/03769; WO 93/19191; WO 94/28938; WO 95/11984 and WO 95/00655.

Other gene delivery vehicles and methods may be employed, including polycationic condensed DNA linked or unlinked to killed adenovirus alone, for example Curiel (1992) Hum. Gene Ther. 3:147-154; ligand linked DNA, for example see Wu (1989) J. Biol. Chem. 264:16985-16987; eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip (1994) Mol. Cell Biol. 14:2411-2418, and in Woffendin (1994) Proc. Natl. Acad. Sci. 91:1581-1585.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Nos. WO 95/13796, WO 94/23697, and WO 91/14445, and EP No. 524 968.

Further non-viral delivery suitable for use includes mechanical delivery systems such as the approach described in Woffendin et al. (1994) Proc. Natl. Acad. Sci. USA 91:11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/11033.

In general, a daily dose of relaxin may be from about 0.1 to 500 μg/kg of body weight per day, from about 6.0 to 200 μg/kg, or from about 12 to 100 μg/kg. In some embodiments, it is desirable to obtain a serum concentration of relaxin at or above about 1.0 ng/ml, from about 0.5 to about 50 ng/ml, from about 1 to about 20 ng/ml. For administration to a 70 kg person, a dosage may be in a range of from about 2 μg to about 2 mg per day, from about 10 μg to 500 μg per day, or from about 50 μg to about 100 μg per day. The amount of relaxin administered will, of course, be dependent on the subject and the severity of the affliction, the manner and schedule of administration and the judgment of the prescribing physician.

In employing relaxin for treatment of diseases relating to cardiac fibrosis any pharmaceutically acceptable mode of administration can be used. Relaxin can be administered either alone or in combination with other pharmaceutically acceptable excipients, including solid, semi-solid, liquid or aerosol dosage forms, such as, for example, tablets, capsules, powders, liquids, gels, suspensions, suppositories, aerosols or the like. Relaxin can also be administered in sustained or controlled release dosage forms (e.g., employing a slow release bioerodable delivery system), including depot injections, osmotic pumps (such as the Alzet implant made by Alza), pills, transdermal and transcutaneous (including electrotransport) patches, and the like, for prolonged administration at a predetermined rate, preferably in unit dosage forms suitable for single administration of precise dosages. The compositions will typically include a conventional pharmaceutical carrier or excipient and relaxin. In addition, these compositions may include other active agents (e.g., other angiogenic agents, other vasodilation-promoting agents), carriers, adjuvants, etc. Generally, depending on the intended mode of administration, the pharmaceutically acceptable composition will contain about 0.1% to 90%, about 0.5% to 50%, or about 1% to about 25%, by weight of relaxin, the remainder being suitable pharmaceutical excipients, carriers, etc. Actual methods of preparing such dosage forms are known, or will be apparent, to those skilled in this art; for example, see Remington's Pharmaceutical Sciences, Mack Publishing Company, Easton, Pa., 15th Edition, 1995. The formulations of human relaxin described in U.S. Pat. No. 5,451,572, are non-limiting examples of suitable formulations which can be used in the methods of the present invention.

Parenteral administration is generally characterized by injection, either subcutaneously, intradermally, intramuscularly or intravenously, or subcutaneously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, and the like.

The percentage of relaxin contained in such parenteral compositions is highly dependent on the specific nature thereof, as well as the needs of the subject. However, percentages of active ingredient of 0.01% to 10% in solution are employable, and will be higher if the composition is a solid which will be subsequently diluted to the above percentages. In general, the composition will comprise 0.2-2% of the relaxin in solution.

Parenteral administration may employ the implantation of a slow-release or sustained-release system, such that a constant level of dosage is maintained. Various matrices (e.g., polymers, hydrophilic gels, and the like) for controlling the sustained release, and for progressively diminishing the rate of release of active agents such as relaxin are known in the art. See, U.S. Pat. No. 3,845,770 (describing elementary osmotic pumps); U.S. Pat. Nos. 3,995,651, 4,034,756 and 4,111,202 (describing miniature osmotic pumps); U.S. Pat. Nos. 4,320,759 and 4,449,983 (describing multichamber osmotic systems referred to as push-pull and push-melt osmotic pumps); and U.S. Pat. No. 5,023,088 (describing osmotic pumps patterned for the sequentially timed dispensing of various dosage units).

Drug release devices suitable for use in administering relaxin according to the methods of the invention may be based on any of a variety of modes of operation. For example, the drug release device can be based upon a diffusive system, a convective system, or an erodible system (e.g., an erosion-based system). For example, the drug release device can be an osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump, are also suitable for use with the present invention. Examples of such devices include those described in, for example, U.S. Pat. Nos. 4,692,147; 4,360,019; 4,487,603; 4,360,019; 4,725,852, and the like. In general, the present treatment methods can be accomplished using any of a variety of refillable, non-exchangeable pump systems. Osmotic pumps have been amply described in the literature. See, e.g., WO 97/27840; and U.S. Pat. Nos. 5,985,305 and 5,728,396.

Relaxin may be administered over a period of hours, days, weeks, or months, depending on several factors, including the severity of the disease being treated, whether a recurrence of the disease is considered likely, etc. The administration may be constant, e.g., constant infusion over a period of hours, days, weeks, months, etc. Alternatively, the administration may be intermittent, e.g., relaxin may be administered once a day over a period of days, once an hour over a period of hours, or any other such schedule as deemed suitable.

Formulations of relaxin may also be administered to the respiratory tract as a nasal or pulmonary inhalation aerosol or solution for a nebulizer, or as a microfine powder for insufflation, alone or in combination with an inert carrier such as lactose, or with other pharmaceutically acceptable excipients. In such a case, the particles of the formulation may advantageously have diameters of less than 50 micrometers, preferably less than 10 micrometers.

Relaxin may be used in conjunction with other therapeutics, and in particular when treating cardiac fibrosis in patients with congestive heart failure or advanced cardiomyopathy. Current therapy for heart failure, including congestive heart failure, focuses on increasing cardiac output without causing undue demands on the myocardium. To achieve these ends, various combinations of diuretics, vasodilators and inotropic agents are used to decrease blood volume, to decrease peripheral resistance, and to increase force of cardiac contraction. Standard of care generally includes beta blockers, angiotensin converting enzyme and/or angiotensin II receptor blockers. Current therapy therefore depends on balancing the effects of multiple drugs to achieve the clinical needs of individual patients, and is plagued by adverse reactions to the drugs used.

LGR7 Activating Antibodies

In a specific embodiment the pharmaceutical is comprised of an antibody that selectively binds to and activates LGR7 to effect the antifibrotic response seen upon administration of relaxin. The antibody for human therapeutic use may be humanized or derived initially from a human source (e.g., phage display). In a preferred embodiment, the LGR7 antibody is a human or humanized monoclonal antibody.

One method for selecting an antibody which preferentially binds to LGR7 is by using a hybridoma which produces a murine monoclonal antibody which preferentially binds to LGR7. These hybridomas provide a reliable source of well-characterized reagents for the construction of antibodies and are particularly useful when their epitope reactivity and affinity has been previously characterized. Another source for such construction includes the use of human monoclonal antibody producing cell lines. (Marasco, W. A., et al., Proc Natl Acad Sci USA, 90:7889-7893 (1993); Chen, S. Y., et al., Proc Natl Acad Sci USA 91:5932-5936 (1994)). Another example includes the use of antibody phage display technology to construct new antibodies against different epitopes on a target molecule. (Burton, D. R., et al., Proc Natl Acad Sci USA 88:10134-10137(1991); Hoogenboom H. R., et al., Immunol Rev 130:41-68 (1992); Winter G., et al., Annu Rev Immunol 12:433-455 (1994); Marks, J. D., et al., J Biol Chem 267: 16007-16010 (1992); Nissim, A., et al., EMBO J 13:692-698 (1994); Vaughan T. J., et al., Nature Bio 14:309-314 (1996); Marks C., et al., New Eng J Med 335:730-733 (1996)). For example, very large naive human sFv libraries have been and can be created to offer a large source or rearranged antibody genes against a plethora of target molecules.

Other sources include transgenic mice that contain a human immunoglobulin locus instead of the corresponding mouse locus as well as stable hybridomas that secrete human antigen-specific antibodies. (Lonberg, N., et al., Nature 368:856-859 (1994); Green, L. L., et al., Nat Genet 7:13-21 (1994)). Such transgenic animals provide another source of human antibody genes through either conventional hybridoma technology or in combination with phage display technology. In vitro procedures to manipulate the affinity and fine specificity of the antigen binding site have been reported including repertoire cloning (Clackson, T., et al., Nature 352:624-628 (1991); Marks, J. D., et al., J Mol Biol 222:581-597 (1991); Griffiths, A. D., et al., EMBO J 12:725-734 (1993)), in vitro affinity maturation (Marks, J. D., et al., Biotech 10:779-783 (1992); Gram H., et al., Proc Natl Acad Sci USA 89:3576-3580 (1992)), semi-synthetic libraries (Hoogenboom, H. R., supra; Barbas, C. F., supra; Akamatsu, Y., et al., J Immunol 151:4631-4659 (1993)) and guided selection (Jespers, L. S., et al., Bio Tech 12:899-903 (1994)). Starting materials for these recombinant DNA based strategies include RNA from mouse spleens (Clackson, T., supra) and human peripheral blood lymphocytes (Portolano, S., et al., supra; Barbas, C. F., et al., supra; Marks, J. D., et al., supra; Barbas, C. F., et al., Proc Natl Acad Sci USA 88: 7978-7982 (1991)) and lymphoid organs and bone marrow from HIV-1-infected donors (Burton, D. R., et al., supra; Barbas, C. F., et al., Proc Natl Acad Sci USA 89:9339-9343 (1992)).

Thus, one can readily screen an antibody to insure that it has a sufficient binding affinity for the LGR7. The binding affinity (K_{d}) should preferably be at least about 10⁻⁷ l/mol, more preferably at least about 10^{-8 l/mol.}

Pharmacophohore Design and the Relaxin-LGR7 Interface.

LGR7 and relaxin form a complex with a particular molecular interaction, and pharmacophores fitting this geometric and chemical description can be used in the present methods to activate LGR7 at the LGR7-relaxin interface. These activators can be used in place of or in addition to relaxin in the presently described methods of the invention. Identifying pharmacophores of the invention requires the identification of small molecules, peptides, and the like that mimics the positive image of the residues that comprise the relaxin binding site on LGR7. A successful compound binds to LGR7, activating and thereby the effects similar to those seen upon relaxin administration.

Assay to Identify Activating Pharmacophores

Candidate molecules as LGR7 activating pharmacophores can encompass numerous chemical classes, including, but not limited to, peptides and small molecules. Candidate pharmacophores can comprise functional groups necessary for structural interaction with proteins, particularly hydrogen bonding, and typically include at least an amine, carbonyl, hydroxyl or carboxyl group, preferably at least two of the functional chemical groups. The candidate pharmacophores often comprise cyclical carbon or heterocyclic structures and/or aromatic or polyaromatic structures substituted with one or more of the above functional groups. Candidate inhibitor pharmacophores are also found among biomolecules including, but not limited to: polynucleotides, peptides, saccharides, fatty acids, steroids, purines, pyrimidines, derivatives, structural analogs or combinations thereof.

Candidate LGR7 activating pharmacophores can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacologically relevant scaffolds may be subjected to directed or random chemical modifications, such as acylation, alkylation, esterification, amidification, etc. to produce structural analogs.

Identification of structural aspects of proteins involved in relaxin-LGR7 complex formation can define a tertiary structure to be used in an assay to design pharmacophores that modulate molecules and/or protein:protein interactions in the complex. Specifically, a dataset of compounds (small molecules, peptides, etc) having a particular tertiary structure can be identified using techniques known in the art, such as medicinal chemistry, combinatorial chemistry and molecular modeling, to determine molecules that are likely to bind to the atoms or groups of atoms of a protein involved in the binding of LGR7 and relaxin. Optionally, factors such as hydrophobicity and hydrophilicity, placement of the functional residues in a structural motif, and the like may also be taken into account.

In a preferred embodiment of the assay of the invention, the assay involves (1) matching compounds in a library with the binding site regarding spatial orientation; (2) screening candidate compounds visually using computer generated molecular display software; and (3) experimentally screening actual compounds against LGR7in the presence and absence of relaxin to determine compounds which enhance signalling activity through LGR7.

Once the functional residues of the target protein are identified, this portion of the molecule can serves as a template for comparison with known molecules, e.g., in a database such as Available Chemicals Database (ACD, Molecular Design Labs, 1997), or it may be used to design molecules de novo. In one example, the initial group of identified molecules may contain tens or hundreds of thousands or more of different non-peptide organic compounds. A different or supplemental group may contain millions of different peptides which could be produced synthetically in chemical reactions or via bacteria or phage. Large peptide libraries and methods of making such are disclosed in U.S. Pat. No. 5,266,684, issued Nov. 30, 1993, and U.S. Pat. No. 5,420,246, issued May 30, 1995, which are incorporated herein by reference. Libraries of non-peptide organic molecules are disclosed in PCT publication WO 96/40202, published Dec. 19, 1996, incorporated herein by reference.

The initial library of molecules is screened via computer generated modeling, e.g., computer models of the compounds are matched against a computer model of the relaxin ligand binding site on LGR7 to find molecules which mimic the spatial orientation and basic structure of the relaxin epitope. This screening should substantially reduce the number of candidate molecules relative to the initial group.

The screened group is then subjected to further screening visually using a suitable computer program which makes viewable images of the molecules. The resulting candidate molecules are then actually tested for their ability to enhance relaxin-LGR7 complex formation and resulting activation of LGR7.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Expression of Relaxin and LGR7 in Cardiac Cells

Distribution of relaxin and LGR7 expression was determined in atrial myocytes and fibroblasts, ventricular myocytes and fibroblasts, and vascular smooth muscle cells (VSMC). These cells were obtained from neonate rats using standard collagenase digestion methods. Neonate (1 day old) Sprague Dawley rats were used for tissue collection and subsequent cardiac cell isolation and preparation. The cells were maintained in DMEM supplemented with 10% fetal calf serum, penicillin (50 U/ml), and streptomycin (50 μg/ml) (DMEM-FBS). Cardiac fibroblast isolation from myocytes and subsequent preparation were performed as described previously (Gray M O et al. (1998) Cardiovasc Res 40:352-363). These preparations contained more than 95% cardiac fibroblasts as determined by morphological appearance and immunocytochemical staining. Fibroblasts were used between passages 2 and 4 for all studies.

Relaxin-1, relaxin-3, and rat LGR7 mRNA expression from neonate atrial and ventricular myocytes and fibroblasts and VSMC was determined by RT-PCR. Freshly isolated cells or cells plated in six-well plates and grown to confluency in DMEM-FBS were used for RNA extraction and analysis. RNA extraction and RT-PCR of relaxin-1 (homologous to human relaxin-2) and LGR7 gene expression in atrial myocytes and fibroblasts, ventricular myocytes and fibroblasts, and VSMCs of rats (n=3-4 samples per cell type) were performed as previously described (Kompa A R, Samuel C S, Summers R J (2002) 137:710-718;Bathgate RAD, et al. (2002) J Biol Chem 277:1148-1157). Rat cardiac cells were screened for relaxin-1 using primer sequences detailed previously (Kompa et al., supra). Relaxin-3 expression was also determined using specific forward (5′-TGCGGAGGCTCACGATGGCGC-3′) and reverse (5′-ATAGCTGACAGCAGGTTGGAC-3′) primers. LGR7 expression was determined using the following primers: forward, 5′-GTGTATCCTTTTCGGTGTTTAAGG-3′; and reverse, 5′-GAATAAGAATTGAGTCTAGATGAC-3′. For relaxin-1 gene expression, an annealing temperature of 55 C (40 cycles) was used; for relaxin-3 expression, an annealing temperature of 58 C (40 cycles) was used; and for LGR7 expression, the following (touch-down) annealing temperatures were used: 54 C (two cycles), 52 C (two cycles), 50 C (two cycles), and 48 C (34 cycles). Glyceraldehyde-3-phosphate dehydrogenase was used in separate PCR reactions to control for quality and equivalent loading of the cDNA. Aliquots of the PCR products were electrophoresed on 2% (wt/vol) agarose gels stained with ethidium bromide and photographed.

Rat relaxin-1 gene transcripts were undetectable in all cell types studied. Relaxin-3 mRNA expression, however, was clearly identified in all atrial and ventricular cells studied, suggesting that it represents the predominant form of relaxin in the rat heart. LGR7 gene expression was detected in freshly isolated atrial and ventricular myocytes but was either not detected (2-day cultured atrial myocytes) or decreased (2-day cultured ventricular fibroblasts) in cultured myocytes. LGR7 was expressed in atrial and ventricular fibroblasts, and this expression persisted during culture (from passages 1-4). LGR7 transcripts were not detected in 2-day cultured VSMC. These findings demonstrated that neonatal cardiac fibroblasts expressed LGR7 and should, therefore, respond to relaxin.

### Example 2

### Relaxin Inhibits TGF-β- and Ang II-Stimulated Collagen Deposition by Reducing Collagen Secretion

The ability of rhRLX to modulate collagen synthesis, degradation, and deposition by cardiac fibroblasts was examined for a number of conditions. rhRLX was kindly provided by the Connetics Corporation (Palo Alto, Calif.), TGF-β was obtained from Bioscientific Australia (Sydney, New South Wales, Australia), and Ang II was obtained from Sigma-Aldrich (St. Louis, Mo.).

It has been demonstrated that Ang II induces TGF-β release from cardiac fibroblasts, and together, they act in synergistic pathways to interfere with normal structure and function of the surrounding myocardium (Chua C C et al. (1994) Biochim Biophys Acta 1223:141-147; Lee A A et al. (1995) J Mol Cell Cardiol 27:2347-2357; Campbell S E, Katwa L C (1997) J Mol Cell Cardiol 29:1947-1958).

Atrial and ventricular fibroblasts were subjected to TGF-β or Ang II in the absence or presence of rhRLX over 3 days. For the TGF-β studies, cells were treated for 72 hours with rhRLX (100 ng/ml), TGF-β (1 or 2 ng/ml), or a combination of rhRLX (100 ng/ml) and TGF-β (2 ng/ml) in serum-free DMEM supplemented with lactalbumin hydrolysate. For the Ang II studies, fibroblasts were treated with Ang II (1×10⁻⁷ M and 5×10⁻⁷ M) or a combination of rhRLX (100 ng/ml) and Ang II (1 or 5×10⁻⁷ M). TGF-B3 (1 ng/ml) alone induced an 85-95% increase in secretion of interstitial (types I and III) collagen into the cultured media. Similarly, Ang II (5×10⁻⁷ M, 1×10⁻⁷ M) alone increased collagen secretion by 15-20% and 50-60% (P<0.05). rhRLX (100 ng/ml) alone had no effect on collagen expression in fibroblasts, as previously described (Unemori E N, Amento E P (1990) J Biol Chem 265:10681-10685; Unemori E N et al. (1996) J Clin Invest 98:2739-2745), but significantly inhibited the TGF-β—(by 60-65%, P<0.05) and Ang II—(by 70-80%, P<0.05) induced effects on collagen secretion over a 72-h period.

Fibroblasts were also plated at 5/mm² and grown for 7 days in the absence or presence of rhRLX (100 ng/ml). The rhRLX dose used in these experiments was based on previous studies on fibroblast cultures, whereby at this concentration, rhRLX demonstrated maximal effects on collagen expression (Unemori E N, Amento E P (1990) J Biol Chem 265:10681-10685; Unemori E N et al. (1996) J Clin Invest 98:2739-2745). Furthermore, the dose of rhRLX used was well within physiological levels of circulating relaxin in pregnant rats, which attain 50-100 ng/ml relaxin from day 14-20 of gestation and up to 180 ng/ml during parturition (Sherwood O D et al. (1980) Endocrinology 107:691-698).

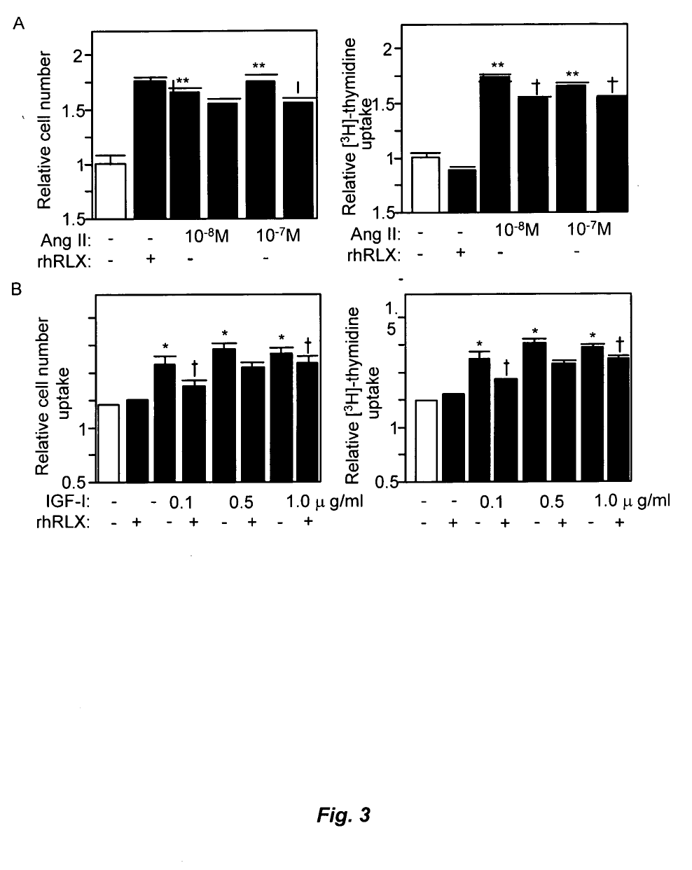

For measurement of collagen synthesis, collagen was biosynthetically labelled with [³H]-proline in the presence of ascorbate and β-aminopropionitrile, as previously described (Unemori et al, supra). For measurement of collagen deposition into the cell matrix, cell layers (per well) were isolated and hydrolyzed with 6 M hydrochloric acid for measurement of hydroxyproline content, as described previously (Samuel C S et al. (1996) Endocrinology 137:3884-3890; Gallop P M, Paz M A (1975) Physiol Rev 55:418487). Hydroxyproline values were then converted to total collagen content by multiplying by a factor of 6.94 (based on hydroxyproline representing approximately 14.4% of the amino acid composition of collagen in most mammalian tissues) (Gallop P M, Paz M A (1975) Physiol Rev 55:418-487). Collagen deposition into the matrix of fibroblasts was up-regulated by TGF-β (65-85%, P<0.05; FIG. 3) and Ang II (20-50%, P<0.05; FIG. 3). As with its effects of collagen secretion into the media, rhRLX (100 ng/ml) alone had no effect on collagen deposition into the matrix when applied to higher-density (atrial and ventricular) cells, but rhRLX significantly decreased collagen deposition stimulated with TGF-β (P<0.05) or Ang II (P<0.05). rhRLX (100 ng/ml) treatment of low-density fibroblast cultures (which accelerate the differentiation of fibroblasts to myofibroblasts) also significantly (P<0.05) inhibited the collagen content of the ECM by 38% compared with the collagen content from untreated cultures.

### Example 3

### Relaxin Increases MMP Expression in Cardiac Cells

The effects of rhRLX on MMP expression, were determined using gelatin zymography of conditioned media, performed as previously described (Talhouk R S et al. (1991) 112:439-449). Briefly, the neonatal cardiac cells (1×10⁵/cm²) were treated with either TGF-β (2 ng/ml) or Ang II (10⁻⁷ M) in the absence or presence of rhRLX (100 ng/ml) for 72 h; the final 24 hours of treatment were under serum-free conditions. Equal aliquots of the collected media were then analyzed on zymogram gels consisting of 7.5% acrylamide and 1 mg/ml gelatin, and the gels were subsequently treated as previously detailed (Talhouk R S, supra). A representative zymograph of MMP-2 and MMP-9 expression is shown in FIG. 3.

TGF-β (2 ng/ml) treatment of cells induced a modest increase in MMP-2 expression over a 72-h period, as detected by gelatin zymography. However, rhRLX (100 ng/ml) treatment of cardiac fibroblasts in the presence of TGF-β (2 ng/ml) significantly increased both the latent (by 55-60%, P<0.01) and active (by 20-25%, P<0.01) forms of MMP-2, while having no marked effects on MMP-9 expression over 72-h of culture. rhRLX also increased MMP-2 expression in the presence of Ang II (by 10-30% of that from untreated cultures, P<0.05) over 3 days in culture.

Collagen content of cell layers from untreated fibroblasts and cells treated with rhRLX (100 ng/ml) alone, TGF-β (2 ng/ml) alone, or TGF-β (2 ng/ml) and rhRLX (100 ng/ml) was measured after 72 hours of culture. The collagen content of cell layers from untreated atrial fibroblasts and cells treated with Ang II (10⁻⁷ M) alone or Ang II (10⁻⁷ M) and rhRLX (100 ng/ml) was measured after 72 hours of culture. Results are presented as the mean±SE of relative collagen content from three to four separate experiments. *, P<0.05; and **, P<0.01 compared with values from untreated cells. †, P<0.05 compared with values from TGF-β-or Ang II-treated cells. Additionally, rhRLX (100 ng/ml) treatment of low-density cells (5/mm²) over 7 days caused an inhibition of collagen deposition. Results are presented as the mean±SE of relative collagen content from three separate experiments (six assays per group from each experiment). \, P<0.05 compared with values from untreated cells.

### Example 4

### Relaxin Inhibits Ang II- and IGF-I-Stimulated Fibroblast Proliferation

The ability of rhRLX to modulate cardiac fibroblast proliferation was investigated in the absence or presence of Ang II and IGF-I, which both potently stimulate fibroblast proliferation. Cardiac fibroblasts were plated at an equal density of 10⁴ cells/96-well plate in DMEM-FBS. Twenty-four hours later, media were changed to DMEM supplemented with lactalbumin hydrolysate, and cells were labelled with [³H]-thymidine (3 μCi/ml) in the presence of rhRLX (100 ng/ml) alone or in combination with IGF-I (0.1 μg/ml, 0.5 μg/ml, and 1 μg/ml) or Ang II (10⁻⁸ M and 10⁻⁷ M) for 72 hours. [³H]-thymidine incorporation and cell counts, using a hemocytometer, were evaluated. Ang II significantly increased atrial and ventricular fibroblast proliferation, as determined by cell counts and [³H]-thymidine incorporation, when administered at concentrations of 10⁻⁸ M (63-75%, P<0.01) and 10⁻⁷ M (6473%, P<0.01) over a 72-h culture period (FIG. 3A). IGF-I also significantly augmented fibroblast proliferation at doses of 0.1 μg/ml (27-30%, P<0.05), 0.5 μg/ml (36-40%, P<0.05), and 1.0 μg/ml (34-40%, P<0.05) over 3 days of culture (FIG. 3B) rhRLX (100 ng/ml) alone had no significant effects on basal fibroblast proliferation but significantly inhibited the Ang II—(by 15-30%, P<0.05; FIG. 3A) and IGF-I—(by 25-50%, P<0.05, FIG. 3B) mediated effects on cell proliferation.

### Example 5

### Relaxin Inhibits TGF-β- and Ang II-Stimulated Fibroblast Differentiation

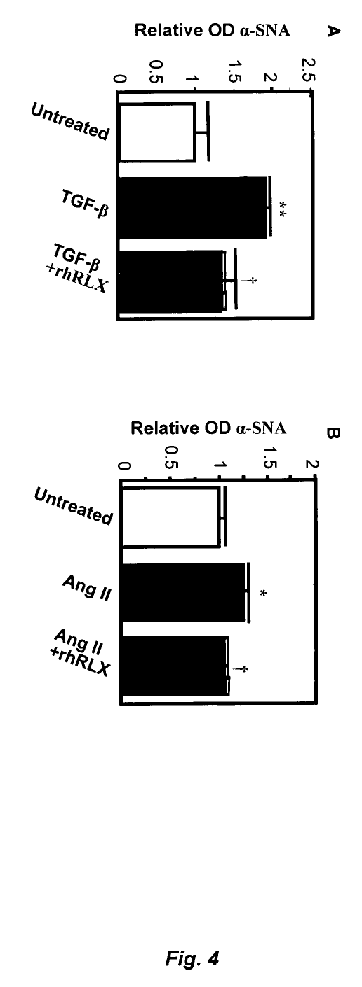

In the normal heart, cardiac fibroblasts are relatively quiescent, but upon pathological stimuli to the heart they proliferate and differentiate into activated myofibroblasts. These myofibroblasts, the hallmarks of which are α-SMA expression and production of prodigious amounts of collagen, are important contributors to fibrosis and the pathology of the heart (Eghbali M (1998) Basic Res Cardiol 87(Suppl 2):183-189; Bouzegrhane F, Thibault G (2002) Cardiovasc Res 53:304-312). Ventricular fibroblast differentiation to myofibroblasts was analyzed by measuring α-SMA expression by ventricular fibroblasts. In all experiments, an equal amount of total protein (10 μg/sample) was analyzed. α-SMA was sized at 43 kDa under nonreducing conditions when detected with a monoclonal antibody. In some experiments, cells were either untreated or treated with TGF-β (2 ng/ml) alone or TGF-β (2 ng/ml) and rhRLX (100 ng/ml) for 72 hours. In other experiments, cells were either untreated or treated with Ang II (10⁻⁷ M) alone or Ang II (10⁻⁷ M) and rhRLX (100 ng/ml) for 72 hours. FIG. 4 shows quantitation of the α-SMA products by densitometry presented as the mean±SE of relative OD α-SMA.

TGF-β (2 ng/ml) and Ang II (10⁻⁷ M) significantly increased fibroblast differentiation, as detected by Western blotting of α-SMA protein. α-SMA expression increased by 85-95% with TGF-β (P<0.01) and 25-35% with Ang II (P<0.05) over 72 hours of culture. Representative figures of duplicate samples from three separate experiments (with either TGF-β or Ang II) and quantitation of the α-SMA products by densitometry is shown in Fi. 4. rhRLX (100 ng/ml) significantly inhibited the TGF-β-induced effects on α-SMA expression (by 65%, P<0.05). Similarly, rhRLX significantly inhibited the Ang II-mediated increase in α-SMA (by 88%, P<0.05; FIG. 4).

### Example 6

### Inhibition of Fibroblast Activation

Fibroblast differentiation into myofibroblasts was determined by α-SMA expression. Ventricular fibroblasts were plated at a density of 2×10⁵ cells/12-well plate and incubated with media (0.7 ml) alone, media containing TGF-β (2 ng/ml) or TGF-β (2 ng/ml), and rhRLX (100 ng/ml) for 72 hours of culture. Collected media were stored at −80 C. Cell layers were trypsinized, and the protein was extracted using Trizol reagent (according to the manufacturer's instructions; Life Technologies, Gaithersburg, Md.) and analyzed by the Bio-Rad dye-binding protein assay (Bio-Rad, Richmond, Calif.).

Protein extracts (in 1% sodium dodecyl sulfate; 10 μg total protein/lane) were electrophoresed under nonreducing conditions on 12.5% acrylamide gels, as previously described (Samuel C S, Sakai L Y, Amento E P (2003) Arch Biochem Biophys 411:47-55). Western blot analysis was performed with a monoclonal antibody to α-SMA (M0851, 1:1000 dilution; Dako Corp., Carpinteria, Calif.) overnight and a goat antimouse IgG secondary antibody (1:2000 dilution; Dako) for 2 hours. Densitometry of the α-SMA bands was performed using a Bio-Rad GS710 Calibrated Imaging Densitometer and Quantity-One software (Bio-Rad).

### Example 7

### Relaxin Inhibits Myocardial Collagen Accumulation and Resulting Fibrosis in vivo in Cardiomyopathy Models

The ability of rhRLX to reduce myocardial collagen accumulation in vivo was investigated in two models of cardiac fibrosis, one due to relaxin disruption (Du X J et al (2003) Cardiovasc Res 57:395-404) and the other to cardiac-restricted overexpression of β₂-AR (Gao X M, et al. (2003) Endocrinology 144:4097-4105). All male relaxin wild-type and relaxin knockout mice used in this study were generated from RLX heterozygous (C57Blk6Jx129SV) parents (Du X J et al. (2003) Cardiovasc Res 57:395-4). The male wild-type and heterozygous β2-AR transgenic mice used were generated from heterozygous (C57Blk6JxSJL) parents (Gao X M, et al. (2003) Endocrinology 144:4097-4105). The animals were housed in a controlled environment and maintained on a 14-hour light, 10-hour dark schedule with access to rodent lab chow (Barastock Stockfeeds, Pakenham, Victoria, Australia) and water.

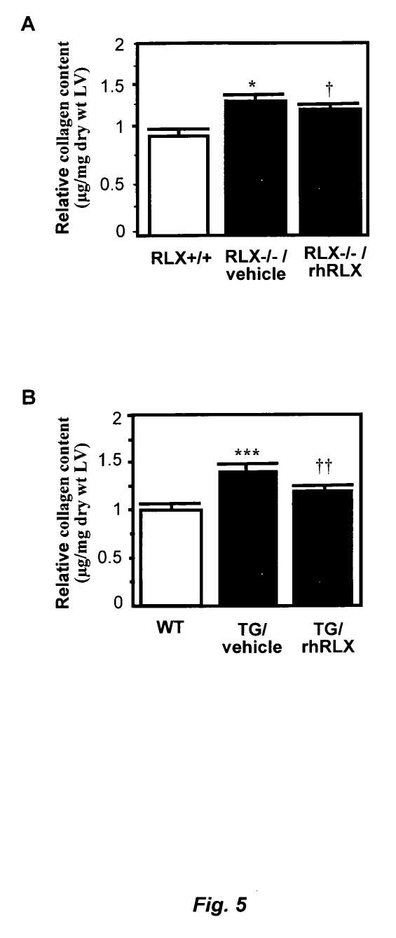

Cardiac fibrosis was established in relaxin-deficient mice by 12-months of age, resulting in a 30% (P<0.05) increase in collagen concentration (FIG. 5A). Myocardial fibrosis was more rapidly established in β₂-AR transgenic mice at 5 months of age, as evidenced by a 60% increase (P<0.001) in collagen concentration (FIG. 5B). This increment in myocardial collagen concentration over respective wild-type control values was significantly reduced by rhRLX (500 μg/kg-d) treatment of animals over 14 days by 40% (P<0.05) in relaxin-deficient mice (FIG. 5A) and by 58% (P<0.01) in β2-AR transgenic mice (FIG. 5B).

Collagen content/dry weight ventricular tissue was determined from 12-month-old relaxin wild-type (RLX+/+) mice (n=8), relaxin knockout (RLX−/−) mice treated with vehicle alone (n=4), and RLX−/− mice treated with 500 μg/kg-day rhRLX (n=4) for 14 days. The results are shown in FIG. 5A. Collagen content/dry weight ventricular tissue was also determined from 5-month-old wild-type (WT) mice (n=8) and β2-AR transgenic (TG) mice treated with vehicle alone (n=8) or with rhRLX (n=8) for 14 days. These results are summarized in FIG. 5B.

The age at which cardiac fibrosis was already established in these animals (12 months of age in male relaxin-deficient mice and 5 months of age in male β₂-AR transgenic mice) were chosen based on observations of these models. For each model, wild-type littermates were used as controls. Osmotic mini-pumps (model 2002; Alza Corp., Cupertino, Calif.), loaded with either vehicle (20 mM sodium acetate buffer, pH 5.0) or rhRLX (500 μg/kg-d) were implanted sc for 14 days which produced circulating relaxin concentrations of 20-40 ng/ml in these animals., and which is within physiological levels of serum relaxin in pregnant rodents (Sherwood O D (1994) Relaxin. In: Knobil E, Neill J D, eds. Physiology of reproduction. 2nd ed. New York: Raven Press; 861-1009; Sherwood O D et al. (1980) Endocrinology 107:691-698). Male relaxin-null mice (n=4 treated with vehicle, n=4 treated with rhRLX), β₂-AR transgenic mice (n=8 treated with vehicle, n=8 treated with rhRLX), and their respective wild-type mice (n=8 for each group) were used for analysis. After 14 days, ventricular tissues were collected and used for hydroxyproline analysis, as previously described (Samuel C S, et al. (1996) Endocrinology 137:3884-3890).

### Example 8

### Relaxin Inhibits Myocardial Collagen Accumulation and Resulting Fibrosis in vivo in a Model of Hypertension

The effects of relaxin administration on the spontaneously hypertensive rat (SHR) model with hypertension-induced cardiac and renal fibrosis and cardiac hypertrophy was examined. The results from this study indicated that relaxin was able to potently and specifically inhibit cardiac and renal fibrosis in this model. All data are expressed as the mean±S.E.M., with p<0.05 described as statistically significant.

Effect of Relaxin on Morphology and Cardiac Function

9-10 month old male SHR and normotensive WKY rats that were used in this study were housed and maintained under standard conditions. Animals were separated into three groups: untreated WKY rats (n=9), vehicle-treated SHR (SHR-V, n=8) and H2-relaxin-treated SHR (SHR-R, n=8). As described previously, rats underwent surgery to subcutaneously implant 14-day ALZET osmotic minipumps (Model No. 2ML2; Durect Corporation, Cupertino, Calif.), loaded either with vehicle (20 mM sodium acetate buffer, pH 5.0; SHR-V) or with 0.5 mg/kg/day H2-relaxin (SHR-R). Samuel C S et al (2004) Kidney Int. (2004) 65:2054-2064; Samuel C S et al. (2003) FASEB J; 17:121-123. Fourteen-day pumps were used to account for the fact that rodents mount antibody responses to exogenous H2 relaxin by −40 days, resulting in increased and variable circulating serum levels. This dose of H2 relaxin was as previously used in the models of cardiomyopathy described in Example 7 above.

Rats were anaesthetized with intraperitoneal administration of ketamine/xylazine/atropine, at 60/12/0.6 mg/kg, respectively. Using SONOS 5500 ultrasound machine and a 12 MHz probe, echocardiography was performed to assess LV systolic and diastolic function immediately before and after the 2-week (H2-relaxin) treatment period. Lijnen P J et al. (2000) Mol Genet Metab 71:418-435. Arterial blood and LV pressures were also assessed by catheterization using a 2Fr Millar catheter immediately after final echocardiography while rats were still anaesthetised, as described previously. Bohle A et al. (1996) Kidney Int. 54(suppl): S2-9. At the end of cardiac functional assessment, hearts and kidneys were harvested for further analysis.

Body weights (BW) were moderately, but significantly lower in SHR groups compared to WKY rats (p<0.05, Table 1). The LV and whole heart weights and LV/BW or heart/BW ratios were significantly higher in SHR-V and SHR-R vs WKY rats (Table 1). Furthermore, measurement of myocyte size showed a slight increase (by 10%) of cardiomyocyte hypertrophy in LVs from SHRs (Table 1). However, this increase was not statistically significant.

Despite the presence of myocardial hypertrophy and fibrosis in SHRs from 3-6 months of age, unequivocal cardiac dysfunction did not occur until 13-18 months of age. With relaxin administration, there was no change in any of the recorded echocardiographic and hemodynamic parameters except for a modest but significant increase in heart rate. Furthermore, the catheter data demonstrated a trend for decreased blood pressure after relaxin treatment. The echocardiography data was analysed using a two-way ANOVA (to account for the two variables: time points and groups used).

Effect of Relaxin on Collagen Content and Subtypes

Total collagen content in the LV myocardium and kidney cortex was determined by quantitative histology and hydroxyproline analysis (see Lijnen, supra). Interstitial collagen content in the LV and kidney was determined by measuring the quantity of picrosirius red and Masson trichrome staining, respectively, as a percentage of the total area within a field. Myocyte cross-sectional area was also obtained from the average of 70-100 cells from randomly selected fields in the LV. The hydroxyproline content of the LV, RV, atria and kidneys was determined as previously described (Samuel C S et al. Kidney Int. (2004) 65:2054-2064; Du X J et al. (2003) Cardiovasc Res. 57:395-404) and then converted to collagen content by multiplying by a factor of 6.94. The results were then expressed as collagen concentration, by dividing the collagen content by the tissue dry weight.

The LV myocardium was also finely diced and the pepsin-digested maturely cross-linked collagen obtained to determine collagen types, by SDS-PAGE as described previously. Samuel C S et al. (1996) Endocrinology (1996); 137:3884-3890.

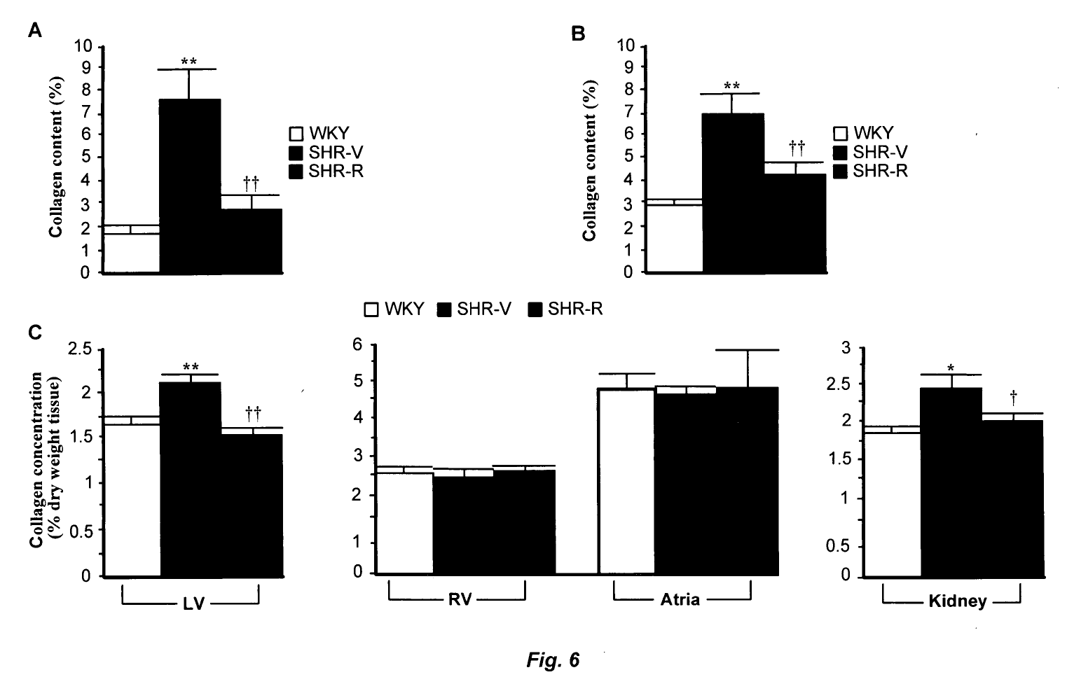

Relative to the WKY group, SHR-V had significantly increased LV collagen content as determined by both methods (p<0.001 and p<0.0l vs WKY, respectively, FIG. 6). 112-relaxin treatment over a 2-week period, significantly reduced this build-up of collagen as detected by histology (p<0.001 vs SHR-V) and hydroxyproline (p<0.01 vs SHR-V, FIG. 6). In contrast to the finding in the LV, there was no significant difference in hydroxyproline content in either the RV or atria, between the three groups (FIG. 6).

The collagen content in the kidney was also significantly increased, relative to the WKY group, as determined by both methods (p<0.001 and p<0.05 vs WKY, respectively, FIG. 6). Similarly, H2-relaxin treatment over 2-weeks normalized renal collagen levels by these assays (both p<0.05 vs SHR-V, FIG. 6.). Over a 14-day period, H2-relaxin significantly reduced the elevated collagen content in the LV and kidney, in particular, types I, III and V collagen. Furthermore, H2-relaxin inhibited fibroblast proliferation and differentiation and induced a significant rise in MMP-2 expression, factors that likely contributed to the reduction in collagen of the LV. The results of this study provide further evidence to support the development of relaxin as a therapeutic agent against cardiac and renal fibrosis, abnormalities induced by hypertension.

Importantly, administration of relaxin to the SHRs, led to a significant decrease in collagen content specifically within the kidney cortex and LV myocardium, and not in the unaffected chambers of the heart. Cardiac injury constitutes an important component of hypertensive syndrome and renal dysfunction acts as an amplifier of the syndrome. Hence, simultaneous and rapid normalization of collagen in both these organs is achieved with the use of relaxin, findings that make relaxin a more desirable intervention to current anti-hypertensive therapies.

Effect of Relaxin on Fibroblast Function and MMP Expression

Proliferation and differentiation of fibroblasts were analysed by Western blotting of PCNA and α-SMA expression, respectively. Cell proliferation and fibroblast differentiation within the LV was examined by Western blot analysis, using monoclonal antibodies to the proliferating cell nuclear antigen (PCNA; Dako Corporation, Carpinteria, Calif.) and α-smooth muscle actin (α-SMA; Dako), as previously described. Samuel C S et al. (2004) Endocrinology 145:4125-4133; Marino T A, et al. (1996) Ant Rec 245:677-684. Western blotting of the house-keeping protein, β-tubulin (monoclonal antibody kindly provided by Dr Zhonglin Chai, Baker H R I, Victoria, Australia), was also performed to demonstrate equal loading of the protein samples.

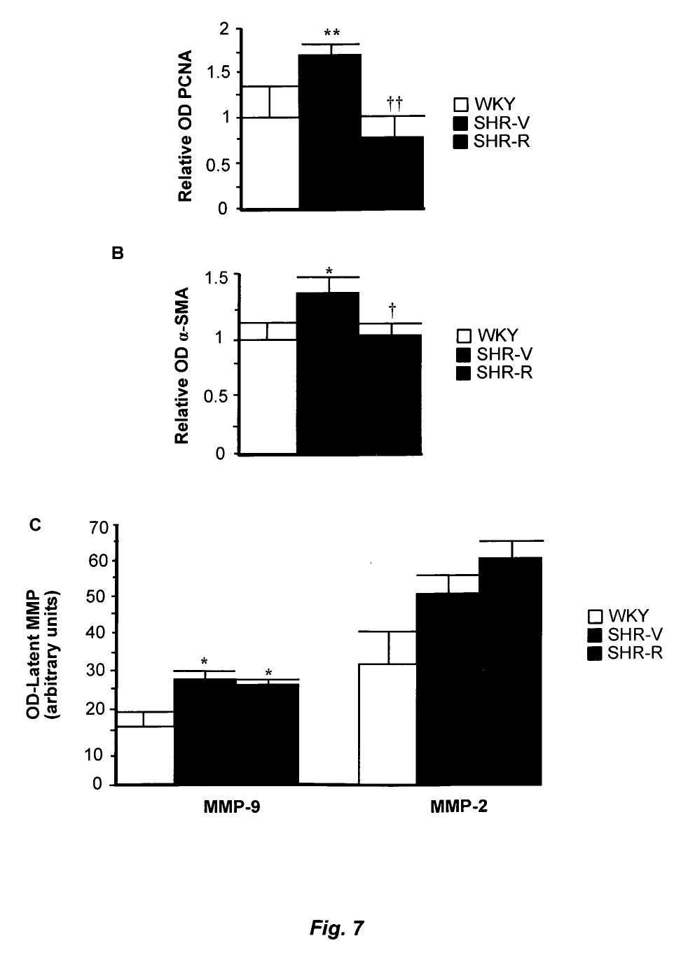

Both fibroblast proliferation (p<0.01 vs WKY) and differentiation (p<0.05 vs WKY) was significantly increased in SHR-V (FIG. 7A & B). Consistent with its ability to inhibit collagen over-expression, H2-relaxin significantly inhibited both PCNA (p<0.0l vs SHR-V and A-SMA expression (p<0.05 vs SFIR-V) over 2-weeks (FIG. 7C).

Gelatin zymography was performed to determine the effect of relaxin on MMP-2 (gelatinase-A) and MMP-9 (gelatinase-B) expression. MMPs were extracted from LV tissue and analysed on zymogram gels as described in Example 3. MMP-2 was the major gelatinase present in the LV of WKY and SHRs, while MMP-9 expression was also detected. In comparison with values from WKY rats, a significant increase in latent MMP-9 expression (p<0.05 vs WKY) and a trend towards increased latent MMP-2 expression were detected in the LV of SHR-V. H2-relaxin treatment of SHRs further increased the expression of latent MMP-2.

Effect of Relaxin on Hypertrophy-Related Gene Expression

Total RNA was extracted from homogenized frozen LV with the use of Trizol® Reagent (Invitrogen, Carlsbad, Calif.) as described by the manufacturer. Following treatment of RNase-free DNase (Promega, NSW, Australia), 1 μg of total RNA was reverse transcribed to cDNA with the use of random primers (Promega) and Superscript III RNase H-Reverse Transcriptase (Invitrogen). Expression of atrial naturetic peptide (ANP) and β-myosin heavy chain (β-MHC), normalised by 18s RNA, were determined by SYBR Green reactions using SYBR Green PCR Master Mix and specific primers with the ABI PRISM 7700 Sequence Detection System.

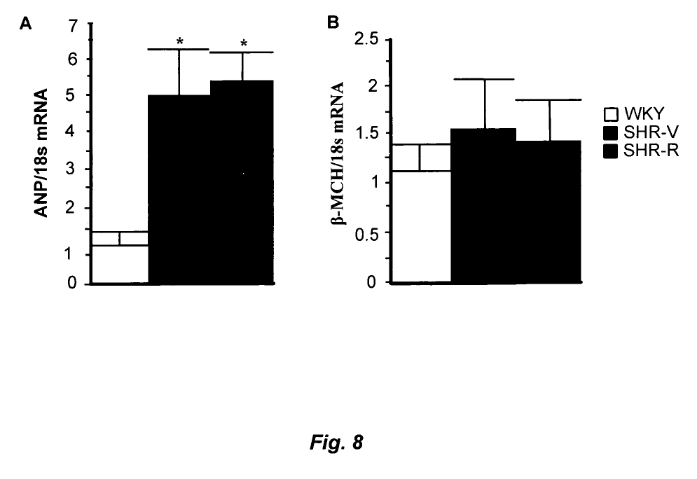

Real-time PCR of hypertrophy-related markers demonstrated that ANP expression was 4-5 fold higher in the SHR-V LV (p<0.05 vs WKY, FIG. 8). However, it was not significantly affected by relaxin treatment (FIG. 8). In contrast, there were no significant differences in β-MHC expression between the three groups studied (FIG. 8).

While the invention has been described in detail, and with reference to specific embodiments thereof, it will be apparent to one of ordinary skill in the art that various changes and modifications can be made therein without departing from the spirit and scope thereof. The invention is thus limited only by the following claims.

1. A method for treating cardiac fibrosis resulting from injury of a mammalian heart comprising the step of contacting a therapeutically effective amount of relaxin and/or an LGR7 activating agent with cardiac cells following the injury in an amount sufficient to reduce the fibrosis.

2. The method of claim 1, wherein the relaxin and/or an LGR7 activating agent is administered systemically.

3. The method of claim 2, wherein the serum concentration of relaxin and/or an LGR7 activating agent is maintained at least about 1 ng/ml.

4. The method of claim 1, wherein the relaxin is administered locally.

5. The method of claim 1, wherein the relaxin and/or an LGR7 activating agent is human relaxin (H2).

6. A method for reducing fibrosis following acute cardiac injury in a patient, the method comprising the step of contacting a therapeutically effective amount of relaxin and/or an LGR7 activating agent with cardiac cells following injury in an amount to decrease cardiac tissue remodelling following administration.

7. The method of claim 6, wherein decrease in remodelling results in a decrease in scar formation resulting from the injury.

8. The method of claim 6, wherein decrease in remodelling results in the patient displaying an increased ejection fraction.

9. A method of protecting tissue in the heart following an ischemic event, the method comprising administration of a relaxin and/or an LGR7 activating agent which prevents collateral damage if tissue surrounding the area damaged by the ischemic event.

10. A method for treating fibrosis in a patient with chronic cardiac disease, the method comprising contacting a therapeutically effective amount of relaxin and/or an LGR7 activating agent with cardiac cells in an amount to prevent fibrosis related to the disease in the cardiac tissue.

11. The method of claim 10, wherein the chronic cardiac disease is cardiomyopathy.

12. The method of claim 10, wherein the chronic cardiac disease is congestive heart failure.

13. A method of inhibiting the activation of fibroblasts in a mammalian heart, comprising administering a relaxin and/or an LGR7 activating agent to a patient, wherein the administering is in a sufficient amount and over a sufficient period of time so as to decrease fibroblast activation in the heart.

14. The method of claim 13, wherein the inhibition results in a decrease of α-SMA expression in cardiac cells.

15. The method according to claim 13 wherein the relaxin is recombinant human relaxin (H2).

16. A method of inhibiting the proliferation of activated fibroblasts in a mammalian heart, comprising administering relaxin and/or an LGR7 activating agent to a patient, wherein the administering is in a sufficient amount and over a sufficient period of time so as to decrease the number of activated fibroblasts in the heart.

17. The method according to claim 16 wherein the relaxin is recombinant human relaxin (H2).

18. A method of antagonizing collagen secretion or deposition in a mammalian heart following injury comprising administering relaxin and/or an LGR7 activating agent to a patient, wherein the administering is in a sufficient amount and over a sufficient period of time to decrease the level of collagen deposition in the heart.

19. The method according to claim 19 wherein the relaxin is recombinant human relaxin (H2).

20. A method of increasing MMP expression and collagen degradation in a mammalian heart following injury comprising administering relaxin and/or an LGR7 activating agent to a patient, wherein the administering is in a sufficient amount and over a sufficient period of time to decrease the level of collagen present in the heart following injury.

21. The method according to claim 20 wherein the relaxin is recombinant human relaxin (H2).
